(19) United States
(12) Patent Application Publication
(10) Pub. No.: US 2021/0401707 A1
(43) Pub. Date: Dec. 30, 2021

(54) AZOXYSTROBIN IN A SULFATE FREE PERSONAL CARE COMPOSITION

(71) Applicant: The Procter & Gamble Company, Cincinnati, OH (US)

(72) Inventors: Eric Scott Johnson, Hamilton, OH (US); Debora W. Chang, Mason, OH (US); Geoffrey Marc Wise, Reading, OH (US); Jeanette Anthea Richards, Liberty Township, OH (US); Brennan Alexander Schilling, Cincinnati, OH (US)

(21) Appl. No.: 17/126,393

(22) Filed: Dec. 18, 2020

Related U.S. Application Data
(60) Provisional application No. 63/044,825, filed on Jun. 26, 2020.

(51) Int. Cl. A61K 8/49; A61Q 5/02; A61Q 5/12; A61K 8/891

(52) U.S. Cl. CPC A61K 8/494; A61Q 5/02; A61K 2800/5426; A61K 8/891; A61K 2800/33; A61Q 5/12

(57) ABSTRACT

The present invention is directed to a personal care composition comprising from about 6% to about 50% of one or more sulfate free surfactants; and from about 0.02% to about 10% of azoxystrobin.

### FIELD OF THE INVENTION

The present invention is directed to azoxystrobin in a sulfate free composition.

### BACKGROUND OF THE INVENTION

Anti-dandruff shampoos have been widely used to treat dandruff and clean hair and scalp with predominately sulfated surfactants. These sulfated surfactants, although clean effectively, may cause irritation to consumers with sensitive scalp skin. Therefore, less irritating surfactants such as sulfate free surfactants, may be a better alternative for antidandruff shampoo formulation. In general, anti-dandruff shampoos are formulated with anti-dandruff agents in combination with surfactants and aqueous systems that are intended to deposit the anti-dandruff agents on the scalp.

### SUMMARY OF THE INVENTION

The present invention is directed to a personal care composition comprising from about 6% to about 50% of one or more sulfate free surfactants; and from about 0.02% to about 10% of azoxystrobin.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography. “QS” means sufficient quantity for 100%.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

### Azoxystrobin and Other Strobilurins

Azoxystrobin, CAS number: 131860-33-8, IUPAC: methyl-(E)-(2-{2[6-(2-cyanophenoxy)-pyrimidin-4-iloxy]-phenyl}-3-methoxyacrylate is an agricultural fungicide belonging to the class of the strobilurins. Strobilurins are either biosynthesized by various Basidiomycete fungi such as Strobilurus tenacellus and Oudemansiella mucida or modeled after natural strobilurins and synthesized with retention of the key β-methoxyacrylate toxophore. Some synthesized strobilurins have a modified toxophore e.g. methyl methoxyiminoacetate or methyl-N-methoxycarbamate. Some synthetic strobilurins are azoxystrobin (CAS number: 131860-33-8), coumoxystrobin (CAS number 850881-70-8), dimoxystrobin (CAS number 149961-52-4), enoxastrobin (CAS number 238410-11-2), fluoxastrobin (CAS number 193740-76-0), kresoxim methyl (CAS number 143390-89M), mandestrobin (CAS number 173662-97-0), metominostrobin (CAS number 133408-50-1), orysastrobin (CAS number 248593-16-0), picoxystrobin (CAS number 117428-22-5), pyraclostrobin (CAS number 175013-18-0), pyraoxystrobin (CAS number 862588-11-2), and trifloxystrobin (CAS number 141517-21-7).

Azoxystrobin and other synthetic strobilurins control a broad spectrum of plant fungal disease and are used heavily in crop protection worldwide. Strobilurins work by inhibition of mitochondrial respiration. The specific mode of action of azoxystrobin and other strobilurins is by binding the ubiquinol oxidizing site (Qo site) in the cytochrome b complex III of the electron transport chain and blocking electron transfer between cytochrome b and cytochrome ci. Other compounds with this specific mode of action include synthetic and naturally occurring derivatives of the key β-methoxyacrylate toxophore known as oudemansins also first isolated from Oudemansiella mucida, synthetic and naturally occurring myxothiazols from myxobacteria such as Myxococcus flavus, stigmatellins from myxobacteria such as Stigmatella aurantica and the synthetic agricultural chemicals famoxadone and fenamidone.

Azoxystrobin as an agricultural fungicide has protectant, curative, eradicant, translaminar and systemic properties and inhibits spore germination and mycelial growth, and also shows antisporulant activity. At labelled application rates, azoxystrobin controls the numerous plant pathogens including Erysiphe graminis, Puccinia spp., Lepiosphaeria nodorum, Septoria tritici and Pyrenophora teres on temperate cereals; Pyricularia oryzae and Rhizoctonia solani on rice; Plasmopara viticola and Uncinula necator on vines; Sphaerotheca fuliginea and Pseudoperonospora cubensis on cucurbitaceae; Phytophthora infestans and Alternaria solani on potato and tomato; Mycosphaerella arachidis, Rhizoctonia solani and Sclerotium rolfsii on peanut; Monilinia spp, and Cladosporium carpophilum on peach; Pythium spp. and Rhizoctonia solani on turf; Mycosphaerella spp. on banana; Cladosporium caryigenum on pecan; Elsinoe fawcetii, Colletotrichum spp. and Guignardia citricarpa on citrus; Colletotrichum spp. and Hemileia vastatrix on coffee. Azoxystrobin is a solid material having low solubility in water.

Some tradenames for azoxystrobin include ABOUND FLOWABLE FUNGICIDE, Aframe, Azoxystar, Azoxyzone, AZteroid 1.65 SC Fungicide, AZURE AGRICULTURAL FUNGICIDE, Endow, QUADRIS FLOWABLE FUNGICIDE, Satori Fungicide, Strobe 2L, and Willowood Azoxy 2SC. Azoxystrobin is commercially available from for example Sigma-Aldrich (St. Louis, Mo.) and Ak Scientific, Inc (Union City, Calif.).

In the present invention, the personal care composition may contain from about 0.02% to about 10% of azoxystrobin; from about 0.05% to about 2% of azoxystrobin; from about 0.1% to about 1% of azoxystrobin.

In the present invention, the personal care composition may contain from about 0.02% to about 10% of a strobilurin; from about 0.05% to about 2% of a strobilurin; from about 0.1% to about 1% of a strobilurin.

In the present invention, the particle size of azoxystrobin may be from about 0.5 microns to about 5 microns; from about 1 micron to about 3 microns.

Detersive Surfactant

The cleansing compositions described herein can include one or more surfactants in the surfactant system. The one or more surfactants can be substantially free of sulfate-based surfactants. As can be appreciated, surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. Suitable surfactants for a cleansing composition can include anionic moieties to allow for the formation of a coacervate with a cationic polymer. The surfactant can be selected from anionic surfactants, amphoteric surfactants, zwitterionic surfactants, non-ionic surfactants, and combinations thereof.

Cleansing compositions typically employ sulfate-based surfactant systems (such as, but not limited to, sodium lauryl sulfate) because of their effectiveness in lather production, stability, clarity and cleansing. The cleansing compositions described herein are substantially free of sulfate-based surfactants. “Substantially free” of sulfate based surfactants as used herein means from about 0 wt % to about 3 wt %, alternatively from about 0 wt % to about 2 wt %, alternatively from about 0 wt % to about 1 wt %, alternatively from about 0 wt % to about 0.5 wt %, alternatively from about 0 wt % to about 0.25 wt %, alternatively from about 0 wt % to about 0.1 wt %, alternatively from about 0 wt % to about 0.05 wt %, alternatively from about 0 wt % to about 0.01 wt %, alternatively from about 0 wt % to about 0.001 wt %, and/or alternatively free of sulfates. As used herein, “free of” means 0 wt %.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of sulfolaurates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

The concentration of the surfactant in the composition should be sufficient to provide the desired cleaning and lather performance. The cleansing composition can comprise a total surfactant level of from about 6% to about 50%, from about 5% to about 35%, a total surfactant level of from about 10% to about 50%, by weight, from about 15% to about 45%, from about 15% to about 22%; from about 16% to about 20%; from about 17% to about 20%; by weight, from about 20% to about 40%, by weight, from about 22% to about 35%, and/or from about 25% to about 30%.

The surfactant system can include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof.

The amino acid based anionic surfactant can be a glutamate, for instance an acyl glutamate.

Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium caproyl glutamate, disodium caproyl glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl Glutamate, sodium olivoyl glutamate, disodium olivoyl glutamate, sodium palmoyl glutamate, disodium palmoyl Glutamate, TEA-cocoyl glutamate, TEA-hydrogenated tallowoyl glutamate, TEA-lauroyl glutamate, and mixtures thereof.

The amino acid based anionic surfactant can be an alaninate, for instance an acyl alaninate. Non-limiting example of acyl alaninates can include sodium cocoyl alaninate, sodium lauroyl alaninate, sodium caproyl alaninate, sodium N-dodecanoyl-l-alaninate and combination thereof.

The amino acid based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, sodium caproyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

The amino acid based anionic surfactant can be a glycinate for instance an acyl glycinate. Non-limiting example of acyl glycinates can include sodium cocoyl glycinate, sodium lauroyl glycinate and combination thereof.

The composition can contain anionic surfactants selected from the group consisting of sulfosuccinates, isethionates, sulfonates, sulfoacetates, sulfolaurates, glucose carboxylates, alkyl ether carboxylates, acyl taurates, lactates, lactylates and mixture thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof.

Non-limiting examples of sulfonates can include alpha olefin sulfonates, linear alkylbenzene sulfonates, alkyl glyceryl sulfonates, sodium laurylgluco sides hydroxypropylsulfonate and combination thereof.

Non-limiting examples of sulfoacetates can include sodium lauryl sulfoacetate, ammonium lauryl sulfoacetate and combination thereof.

Non-limiting examples of sulfolaurates can include sodium methyl-2 sulfolaurate, disodium sulfolaurate and combinations thereof.

Non-limiting example of glucose carboxylates can include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof.

Non-limiting example of acyl taurates can include sodium methyl cocoyl taurate, sodium methyl lauroyl taurate, sodium caproyl methyltaurate, sodium methyl oleoyl taurate and combination thereof.

Non-limiting example of lactates can include sodium lactate.

Non-limiting examples of lactylates can include sodium lauroyl lactylate, sodium cocoyl lactylate, and combination thereof.

The surfactant system may further comprise one or more amphoteric surfactants and the amphoteric surfactant can be selected from the group consisting of betaines, propionates, sultaines, hydroxysultaines, amphohydroxypropyl sulfonates, alkyl amphoacetates, alkyl amphodiacetates, alkyl and combination thereof.

Examples of betaine surfactants can include coco dimethyl carboxymethyl betaine, cocoamidopropyl betaine (CAPB), coco-betaine, cetyl betaine, lauryl amidopropyl betaine (LAPB), oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

Examples of propionate surfactants can include sodium cocaminopropionate, sodium cocoamphopropionate, sodium cornamphopropionate, sodium lauraminopropionate, sodium lauroamphopropionate, sodium lauriminodipropionate, disodium capryloamphodipriopionate, disodium cocoamphodipropionate, disodium lauroamphodipropionate, disodium oleoamphodipropionate and combinations thereof.

Non-limiting example of alkylamphoacetates can include sodium cocoamphoacetate, sodium lauroamphoacetate, disodium cocoamphodiacetate and combination thereof.

The amphoteric surfactant can comprise cocamidopropyl betaine (CAPB), lauramidopropyl betaine (LAPB), and combinations thereof.

The cleansing composition can comprise an amphoteric surfactant level from about 0.25 wt % to about 20 wt %, from about 0.5 wt % to about 15 wt %, from about 2 wt % to about 13 wt %, from about 3 wt % to about 15 wt %, and/or from about 5 wt % to about 10 wt %.

The surfactant system may further comprise one or more non-ionic surfactants and the non-ionic surfactant can be selected from the group consisting alkyl polyglucoside, alkyl glycoside, acyl glucamide, alkanolamides, alkoxylated amides, glyceryl esters and mixture thereof.

Non-limiting examples of alkyl polyglucosides can include decyl glucoside, coco-glucoside, lauryl glucoside and combination thereof.

Non-limiting examples of acyl glucamide can include lauroyl/myristoyl methyl glucamide, capryloyl/caproyl methyl glucamide, cocoyl methyl glucamide and combinations thereof.

Non-limiting examples of alkanolamides can include Cocamide MEA, Cocamide DEA, Cocamide, Cocamide Methyl MEA, Cocamide MIPA, Lauramide DEA, Lauramide MEA and combinations thereof.

Non-limiting examples of alkoxylated amides can include PPG-2 Cocamide, PPG-2 Hydroxyethyl Cocamide, PPG-2 Hydroxyethyl Isostearamide and combinations thereof.

Non-limiting examples of glyceryl esters can include glyceryl caprylate, glyceryl caprate, glyceryl cocoate, glyceryl laurate, glyceryl oleate, glyceryl monostearate and combinations thereof.

The present invention may have from about 0.25% to about 20% of one or more amphoteric, nonionic or zwitterionic co-surfactants.

The present invention may have a pH of from about 4 to about 7; from about 5 to about 6.5; from about 5 to about 6; from about 5.5 to about 6; or from about 4.7 to about 5.5.

### Aqueous Carrier

The personal care composition comprises an aqueous carrier. Accordingly, the formulations of the personal care composition can be in the form of pourable liquids (under ambient conditions). Such compositions will therefore typically comprise an aqueous carrier, which is present at a level of from about 20 wt. % to about 95 wt. %, or from about 60 wt. % to about 85 wt. %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carriers useful in the personal care composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, dipropylene glycol, hexylene glycol, glycerin, and propane diol.

### Emulsifiers

In cases where the personal care composition does not include a gel matrix, the 1,2-diol can be pre-emulsified before it is added in the personal care composition. Emulsifiers selection for each conditioning active is guided by the Hydrophilic-Lipophilic-Balance value (HLB value) of emulsifiers. Suitable range of HLB value is 6-16, or suitable range of HLB value is 8-14. Emulsifiers with an HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers.

Rheology Modifier/Thickener

The personal care compositions mentioned above may also contain one or more rheology modifier/thickener to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. Further, the leave-on treatment may comprise from about 0.01% to about 3% of a rheology modifier, alternatively from about 0.1% to about 1% of a rheology modifier,

The one or more rheology modifier may be selected from the group consisting of polyacrylamide thickeners, cationically modified polysaccharides, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and)polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E 10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

A non exclusive list of suitable thickeners for use herein include xanthan, guar, hydroxypropyl guar, scleroglucan, methyl cellulose, ethyl cellulose (commercially available as Aquacote (Registered trademark), hydroxyethyl cellulose (Natrosol (Registered trademark), carboxymethyl cellulose, hydroxypropylmethyl cellulose, microcrystalline cellulose, hydroxybutylmethyl cellulose, hydroxypropyl cellulose (Klucel (Registered trademark), hydroxyethyl ethyl cellulose, cetyl hydroxyethyl cellulose (Natrosol (Registered trademark Plus 330), N-vinylpyrollidone (Povidone (Registered trademark), Acrylates/Ceteth-20 Itaconate Copolymer (Structure (Registered trademark 3001), hydroxypropyl starch phosphate (Structure (Registered trademark ZEA), polyethoxylated urethanes or polycarbamyl polyglycol ester (e.g. PEG-150/Decyl/SMDI copolymer=Aculyn (Registered trademark 44, PEG-150/Stearyl/SMDI copolymer=Aculyn 46 (Registered trademark), trihydroxystearin (Thixcin (Registered trademark) acrylates copolymer (e.g. Aculyn (Registered trademark 33) or hydrophobically modified acrylate copolymers (e.g. Acrylates/Steareth-20 Methacrylate Copolymer=Aculyn (Registered trademark 22), and fatty alcohols, such as cetyl and stearyl alcohol, and combinations thereof.

A. Cationic Polymers

The personal care composition also comprises a cationic polymer. These cationic polymers can include at least one of (a) a cationic guar polymer, (b) a cationic non-guar galactomannan polymer, (c) a cationic tapioca polymer, (d) a cationic copolymer of acrylamide monomers and cationic monomers, and/or (e) a synthetic, non-crosslinked, cationic polymer, which may or may not form lyotropic liquid crystals upon combination with the detersive surfactant (f) a cationic cellulose polymer. Additionally, the cationic polymer can be a mixture of cationic polymers.

The personal care composition may comprise a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure should be sufficient to provide the requisite cationic charge density described above.

The cationic polymer may be, including but not limited to a cationic guar polymer, has a weight average Molecular weight of less than 2 2 million g/mol, or from about 150 thousand to about 2.2 million g/mol, or from about 200 thousand to about 2 2 million g/mol, or from about 300 thousand to about 1.2 million g/mol, or from about 750,000 thousand to about 1 million g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.8 meq/g.

The cationic polymers may have a molecular weight in the range of about 50,000 to less than or equal to 1.8 million and a charge density of about 0.5 to about 1.7 meq/g. The cationic polymer may be in the range of about 100,000 to about 1 million, in the range of about 500,000 to about 1.2 million. The cationic polymer may have a charge density of about 0.6 to about 1.2 meq/g; from about 0.8 to about 1.0 meq/g.

The cationic guar polymer may have a weight average Molecular weight of less than about 1.5 million g/mol, and has a charge density of from about 0.1 meq/g to about 2.5 meq/g. The cationic guar polymer may have a weight average molecular weight of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol or from about 150 thousand to about 800 thousand g/mol, or from about 200 thousand to about 700 thousand g/mol, or from about 300 thousand to about 700 thousand g/mol, or from about 400 thousand to about 600 thousand g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.5 meq/g.

The cationic guar polymer may be formed from quaternary ammonium compounds. The quaternary ammonium compounds for forming the cationic guar polymer may conform to the general formula 1:

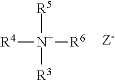

wherein where R³, R⁴ and R⁵ are methyl or ethyl groups; R⁶ is either an epoxyalkyl group of the general formula 2:

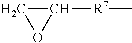

or R⁶ is a halohydrin group of the general formula 3:

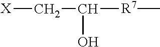

wherein R⁷ is a C₁ to C₃ alkylene; X is chlorine or bromine, and Z is an anion such as Cl-, Br-, I- or HSO₄—.

The cationic guar polymer may conform to the general formula 4:

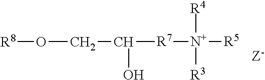

wherein R⁸ is guar gum; and wherein R⁴, R⁵, R⁶ and R⁷ are as defined above; and wherein Z is a halogen. The cationic guar polymer may conform to Formula 5:

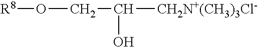

Suitable cationic guar polymers include cationic guar gum derivatives, such as guar hydroxypropyltrimonium chloride. The cationic guar polymer may be a guar hydroxypropyltrimonium chloride. Specific examples of guar hydroxypropyltrimonium chlorides include the Jaguar® series commercially available from Solvay, for example Jaguar® C-500, commercially available from Solvay. Jaguar® C-500 has a charge density of 0.8 meq/g and a molecular weight of 500,000 g/mol. Other suitable guar hydroxypropyltrimonium chloride are: guar hydroxypropyltrimonium chloride which has a charge density of about 1.3 meq/g and a molecular weight of about 500,000 g/mol and is available from Solvay as Jaguar® Optima. Other suitable guar hydroxypropyltrimonium chloride are: guar hydroxypropyltrimonium chloride which has a charge density of about 0.7 meq/g and a molecular weight of about 1,500,000 g/mol and is available from Solvay as Jaguar® Excel. Other suitable guar hydroxypropyltrimonium chloride are: guar hydroxypropyltrimonium chloride which has a charge density of about 1.1 meq/g and a molecular weight of about 500,000 g/mol and is available from ASI, a charge density of about 1.5 meq/g and a molecular weight of about 500,000 g/mole is available from ASI.

Other suitable guar hydroxypropyltrimonium chloride are: Hi-Care 1000, which has a charge density of about 0.7 meq/g and a Molecular weight of about 600,000 g/mole and is available from Solvay; N-Hance 3269 and N-Hance 3270, which have a charge density of about 0.7 meq/g and a molecular weight of about 425,000 g/mol and are available from ASI; N-Hance 3196, which has a charge density of about 0.8 meq/g and a molecular weight of about 1,100,000 g/mol and is available from ASI. AquaCat CG518 has a charge density of about 0.9 meq/g and a Molecular weight of about 50,000 g/mol and is available from ASI. BF-13, which is a borate (boron) free guar of charge density of about 1 meq/g and molecular weight of about 800,000 and BF-17, which is a borate (boron) free guar of charge density of about 1.5 meq/g and molecular weight of about 800,000, and both are available from ASI.

The personal care compositions of the present invention may comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. Non Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The non-guar galactomannan polymer derivatives may have a M. Wt. from about 1,000 to about 10,000,000, and/or from about 5,000 to about 3,000,000.

The personal care compositions of the invention can also include galactomannan polymer derivatives which have a cationic charge density from about 0.5 meq/g to about 7 meq/g. The galactomannan polymer derivatives can have a cationic charge density from about 1 meq/g to about 5 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general formulas 1-5, as defined above.

Cationic non-guar galactomannan polymer derivatives formed from the reagents described above are represented by the general formula 6:

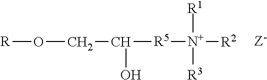

wherein R is the gum. The cationic galactomannan derivative can be a gum hydroxypropyltrimethylammonium chloride, which can be more specifically represented by the general formula 7:

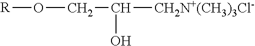

Alternatively, the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose is greater than about 4:1, a molecular weight of about 1,000 g/mol to about 10,000,000 g/mol, and/or from about 50,000 g/mol to about 1,000,000 g/mol, and/or from about 100,000 g/mol to about 900,000 g/mol, and/or from about 150,000 g/mol to about 400,000 g/mol and a cationic charge density from about 1 meq/g to about 5 meq/g, and/or from 2 meq/g to about 4 meq/g and can be derived from a cassia plant.

The personal care compositions can comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cationically modified starch polymers disclosed herein have a percent of bound nitrogen of from about 0.5% to about 4%.

The cationically modified starch polymers for use in the personal care compositions can have a molecular weight about 850,000 g/mol to about 1,500,000 g/mol and/or from about 900,000 g/mol to about 1,500,000 g/mol.

The personal care compositions can include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable.sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures thereof.

The cationically modified starch polymers can be selected from degraded cationic maize starch, cationic tapioca, cationic potato starch, and mixtures thereof. Alternatively, the cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance of about 80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Suitable cationically modified starch for use in personal care compositions are available from known starch suppliers. Also suitable for use in personal care compositions are nonionic modified starch that can be further derivatized to a cationically modified starch as is known in the art. Other suitable modified starch starting materials may be quaternized, as is known in the art, to produce the cationically modified starch polymer suitable for use in personal care compositions.

Starch Degradation Procedure: a starch slurry can be prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

The personal care composition can comprise a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

The cationic copolymer can comprise:
(i) an acrylamide monomer of the following Formula AM:

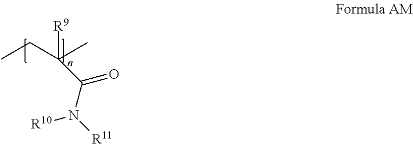

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM:

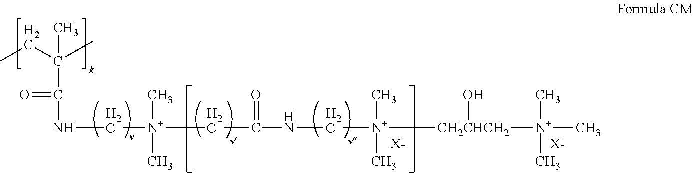

where k=1, each of v, v′, and v″ is independently an integer of from 1 to 6, w is zero or an integer of from 1 to 10, and X⁻ is an anion.

The cationic monomer can conform to Formula CM and where k=1, v=3 and w=0, z=1 and X⁻ is Cl⁻ to form the following structure:

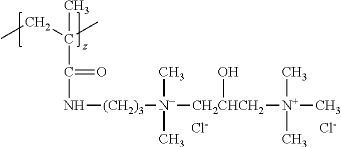

The above structure may be referred to as diquat. Alternatively, the cationic monomer can conform to Formula CM and wherein v and v″ are each 3, v′=1, w=1, y=1 and X⁻ is Cl⁻, such as:

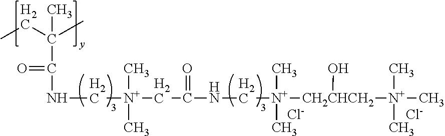

The above structure may be referred to as triquat.

Suitable acrylamide monomer include, but are not limited to, either acrylamide or methacryl amide.

The cationic copolymer (b) can be AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′-pentamethyl-, trichloride. AM:TRIQUAT is also known as polyquaternium-76 (PQ76). AM:TRIQUAT may have a charge density of 1.6 meq/g and a molecular weight of 1.1 million g/mol.

The cationic copolymer may be of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can comprise a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be water-soluble. The cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. The cationized esters of the (meth)acrylic acid containing a quaternized N atom may be quaternized dialkylaminoalkyl (meth)acrylates with C1 to C3 in the alkyl and alkylene groups. Suitable cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom may be dimethylaminoethyl acrylate, which is quaternized with an alkyl halide, or with methyl chloride or benzyl chloride or dimethyl sulfate (ADAME-Quat). the cationic monomer when based on (meth)acrylamides can be quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

Suitable cationic monomer based on a (meth)acrylamide include quaternized dialkylaminoalkyl(meth)acrylamide with C1 to C3 in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, all monomers that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

The cationic copolymer can have a molecular weight from about 100 thousand g/mol to about 1.5 million g/mol, or from about 300 thousand g/mol to about 1 5 million g/mol, or from about 500 thousand g/mol to about 1 5 million g/mol, or from about 700 thousand g/mol to about 1.0 million g/mol, or from about 900 thousand g/mol to about 1.2 million g/mol.

The cationic copolymer can be a trimethylammoniopropylmethacrylamide chloride-N-Acrylamide copolymer, which is also known as AM:MAPTAC. AM:MAPTAC may have a charge density of about 1.3 meq/g and a molecular weight of about 1.1 million g/mol. The cationic copolymer can be AM:ATPAC. AM:ATPAC can have a charge density of about 1.8 meq/g and a molecular weight of about 1.1 million g/mol.

(a) Cationic Synthetic Polymers

The personal care composition can comprise a cationic synthetic polymer that may be formed from

i) one or more cationic monomer units, and optionally

ii) one or more monomer units bearing a negative charge, and/or

iii) a nonionic monomer,

wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers having the following structure:

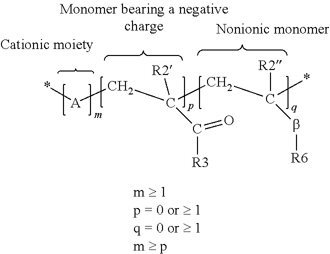

where A, may be one or more of the following cationic moieties:

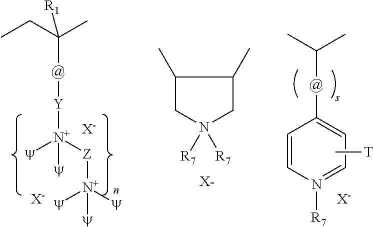

where @=amido, alkylamido, ester, ether, alkyl or alkylaryl;
where Y=C1-C22 alkyl, alkoxy, alkylidene, alkyl or aryloxy;
where ψ=C1-C22 alkyl, alkyloxy, alkyl aryl or alkyl arylox;
where Z=C1-C22 alkyl, alkyloxy, aryl or aryloxy;
where R1=H, C1-C4 linear or branched alkyl;
where s=0 or 1, n=0 or ≥1;
where T and R7=C1-C22 alkyl; and
where X-=halogen, hydroxide, alkoxide, sulfate or alkylsulfate.

Where the monomer bearing a negative charge is defined by R2′=H, C1-C4 linear or branched alkyl and R3 as:

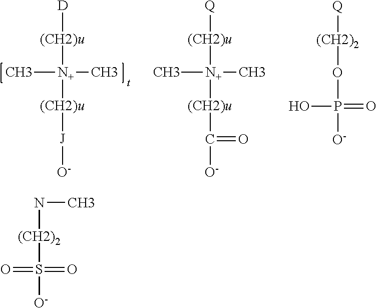

where D=O, N, or S;
where Q=NH₂ or O;
where u=1-6;
where t=0-1; and
where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and 13 is defined as

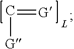

and
where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X-) in association with the synthetic cationic polymers may be any known counterion so long as the polymers remain soluble or dispersible in water, in the personal care composition, or in a coacervate phase of the personal care composition, and so long as the counterions are physically and chemically compatible with the essential components of the personal care composition or do not otherwise unduly impair product performance, stability or aesthetics. Non limiting examples of such counterions include halides (e.g., chlorine, fluorine, bromine, iodine), sulfate and methylsulfate.

The cationic polymer described herein can aid in providing damaged hair, particularly chemically treated hair, with a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer returns the hair to a generally virgin-like, healthier state. Lyotropic liquid crystals are formed by combining the synthetic cationic polymers described herein with the aforementioned anionic detersive surfactant component of the personal care composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in WO 94/06403 to Reich et al. The synthetic polymers described herein can be formulated in a stable personal care composition that provides improved conditioning performance, with respect to damaged hair.

Cationic synthetic polymers that can form lyotropic liquid crystals have a cationic charge density of from about 2 meq/gm to about 7 meq/gm, and/or from about 3 meq/gm to about 7 meq/gm, and/or from about 4 meq/gm to about 7 meq/gm. The cationic charge density may be about 6.2 meq/gm. The polymers also have a M. Wt. of from about 1,000 to about 5,000,000, and/or from about 10,000 to about 1,500,000, and/or from about 100,000 to about 1,500,000.

In the invention cationic synthetic polymers that provide enhanced conditioning and deposition of benefit agents but do not necessarily form lyotropic liquid crystals may have a cationic charge density of from about 0.7 meq/gm to about 7 meq/gm, and/or from about 0.8 meq/gm to about 5 meq/gm, and/or from about 1.0 meq/gm to about 3 meq/gm. The polymers may also have a M. Wt. of from about 1,000 to about 1,500,000, from about 10,000 to about 1,500,000, and from about 100,000 to about 1,500,000.

Suitable cationic cellulose polymers are salts of hydroxyethyl cellulose reacted with trimethyl ammonium substituted epoxide, referred to in the industry (CTFA) as Polyquaternium-10 and available from Dow/Amerchol Corp. (Edison, N.J., USA) in their Polymer LR, JR, and KG series of polymers. Non-limiting examples include: JR-30M, KG-30M, JP, LR-400 and mixtures thereof. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium-24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium-67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

The concentration of the cationic polymers ranges about 0.025% to about 5%, from about 0.1% to about 3%, from about 0.1% to about 1.2%, from about 0.2% to about 1%, from about 0.6% to about 0.9%, by weight of the personal care composition.

1. Water Miscible Solvents

The carrier of the personal care composition may include water and water solutions of lower alkyl alcohols, polyhydric alcohols, ketones having from 3 to 4 carbons atoms, C1-C6 esters of C1-C6 alcohols, sulfoxides, amides, carbonate esters, ethoxylated and proposylated C1-C10 alcohols, lactones, pyrollidones, and mixtures thereof. Non-limited lower alkyl alcohol examples are monohydric alcohols having 1 to 6 carbons, such as ethanol and isopropanol. Non-limiting examples of polyhydric alcohols useful herein include propylene glycol, dipropylene glycol, butylenes glycol, hexylene glycol, glycerin, propane diol and mixtures thereof.

In present invention, the personal care composition may comprise a hydrotrope/viscosity modifier which is an alkali metal or ammonium salt of a lower alkyl benzene sulphonate such as sodium xylene sulphonate, sodium cumene sulphonate or sodium toluene sulphonate.

In the present invention, the personal care composition may comprise silicone/PEG-8 silicone/PEG-9 silicone/PEG-n silicone/silicone ether (n could be another integer), non-limiting examples include PEGS-dimethicone A208) MW 855, PEG 8 Dimethicone D208 MW 2706.

B. Scalp Health Agents

In the present invention, one or more scalp health agent may be added to provide scalp benefits in addition to the anti-fungal/anti-dandruff efficacy provided by the surfactant soluble anti-dandruff agents. This group of materials is varied and provides a wide range of benefits including moisturization, barrier improvement, anti-fungal, anti-microbial and anti-oxidant, anti-itch, and sensates, and additional anti-dandruff agents. Such scalp health agents include but are not limited to: vitamin E and F, salicylic acid, niacinamide, caffeine, panthenol, zinc oxide, zinc carbonate, basic zinc carbonate, glycols, glycolic acid, PCA, PEGs, erythritol, glycerin, triclosan, lactates, hyaluronates, allantoin and other ureas, betaines, sorbitol, glutamates, xylitols, menthol, menthyl lactate, iso cyclomone, benzyl alcohol, a compound comprising the following structure:

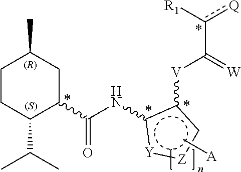

R₁ is selected from H, alkyl, amino alkyl, alkoxy;
Q=H₂, O, —OR₁; —N(R₁)₂; —OPO(OR₁)ₓ; —PO(OR₁)ₓ; —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic
CH, or heteroatom;
A=lower alkoxy, lower alkylthio, aryl, substituted aryl or fused aryl; and stereochemistry is variable at the positions marked*.

and natural extracts/oils including peppermint, spearmint, argan, jojoba and aloe.

C. Optional Ingredients

In the present invention, the personal care composition may further comprise one or more optional ingredients, including benefit agents. Suitable benefit agents include, but are not limited to conditioning agents, cationic polymers, silicone emulsions, anti-dandruff agents, gel networks, chelating agents, and natural oils such as sunflower oil or castor oil. Additional suitable optional ingredients include but are not limited to perfumes, perfume microcapsules, colorants, particles, anti-microbials, foam busters, anti-static agents, rheology modifiers and thickeners, suspension materials and structurants, pH adjusting agents and buffers, preservatives, pearlescent agents, solvents, diluents, anti-oxidants, vitamins and combinations thereof. In the present invention, a perfume may be present from about 0.5% to about 7%.

Such optional ingredients should be physically and chemically compatible with the components of the composition, and should not otherwise unduly impair product stability, aesthetics, or performance The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

1. Conditioning Agents

The conditioning agent of the personal care compositions can be a silicone conditioning agent. The silicone conditioning agent may comprise volatile silicone, non-volatile silicone, or combinations thereof. The concentration of the silicone conditioning agent typically ranges from about 0.01% to about 10%, by weight of the composition, from about 0.1% to about 8%, from about 0.1% to about 5%, and/or from about 0.2% to about 3%. Non-limiting examples of suitable silicone conditioning agents, and optional suspending agents for the silicone, are described in U.S. Reissue Pat. No. 34,584, U.S. Pat. Nos. 5,104,646, and 5,106,609, which descriptions are incorporated herein by reference.

The silicone conditioning agents for use in the compositions of the present invention can have a viscosity, as measured at 25° C., from about 20 to about 2,000,000 centistokes (“csk”), from about 1,000 to about 1,800,000 csk, from about 10,000 to about 1,500,000 csk, and/or from about 20,000 to about 1,500,000 csk.

The dispersed silicone conditioning agent particles typically have a volume average particle diameter ranging from about 0.01 micrometer to about 60 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

Silicone emulsions suitable for use in the present invention may include, but are not limited to, emulsions of insoluble polysiloxanes prepared in accordance with the descriptions provided in U.S. Pat. Nos. 6,316,541 or 4,476,282 or U.S. Patent Application Publication No. 2007/0276087. Accordingly, suitable insoluble polysiloxanes include polysiloxanes such as alpha, omega hydroxy-terminated polysiloxanes or alpha, omega alkoxy-terminated polysiloxanes having an internal phase viscosity from about 5 csk to about 500,000 csk. For example, the insoluble polysiloxane may have an internal phase viscosity less 400,000 csk, may be less than 200,000 csk, may be from about 10,000 csk to about 180,000 csk. The insoluble polysiloxane can have an average particle size within the range from about 10 nm to about 10 micron. The average particle size may be within the range from about 15 nm to about 5 micron, from about 20 nm to about 1 micron, or from about 25 nm to about 500 nm.

The average molecular weight of the insoluble polysiloxane, the internal phase viscosity of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscometer with spindle 6 at 2.5 rpm. The silicone emulsion may further include an additional emulsifier together with the anionic surfactant,

Other classes of silicones suitable for use in compositions of the present invention include but are not limited to: i) silicone fluids, including but not limited to, silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

The conditioning agent of the personal care compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

Gel Network

In the present invention, a gel network may be present. The gel network component of the present invention comprises at least one fatty amphiphile. As used herein, “fatty amphiphile” refers to a compound having a hydrophobic tail group as defined as an alkyl, alkenyl (containing up to 3 double bonds), alkyl aromatic, or branched alkyl group of C12-C70 length and a hydrophilic head group which does not make the compound water soluble, wherein the compound also has a net neutral charge at the pH of the shampoo composition.

The shampoo compositions of the present invention comprise fatty amphiphile as part of the pre-formed dispersed gel network phase in an amount from about 0.05% to about 14%, may be from about 0.5% to about 10%, and may be from about 1% to about 8%, by weight of the shampoo composition.

According to the present invention, suitable fatty amphiphiles, or suitable mixtures of two or more fatty amphiphiles, have a melting point of at least about 27° C. The melting point, as used herein, may be measured by a standard melting point method as described in U.S. Pharmacopeia, USP-NF General Chapter <741>“Melting range or temperature”. The melting point of a mixture of two or more materials is determined by mixing the two or more materials at a temperature above the respective melt points and then allowing the mixture to cool. If the resulting composite is a homogeneous solid below about 27° C., then the mixture has a suitable melting point for use in the present invention. A mixture of two or more fatty amphiphiles, wherein the mixture comprises at least one fatty amphiphile having an individual melting point of less than about 27° C., still is suitable for use in the present invention provided that the composite melting point of the mixture is at least about 27° C.

Suitable fatty amphiphiles of the present invention include fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxyalted amines, fatty carbamates, fatty amine oxides, fatty acids, alkoxylated fatty acids, fatty diesters, fatty sorbitan esters, fatty sugar esters, methyl glucoside esters, fatty glycol esters, mono, di & tri glycerides, polyglycerine fatty esters, alkyl glyceryl ethers, propylene glycol fatty acid esters, cholesterol, ceramides, fatty silicone waxes, fatty glucose amides, and phospholipids and mixtures thereof.

In the present invention, the shampoo composition may comprise fatty alcohol gel networks. These gel networks are formed by combining fatty alcohols and surfactants in the ratio of from about 1:1 to about 40:1, from about 2:1 to about 20:1, and/or from about 3:1 to about 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

The fatty alcohol can be included in the fatty alcohol gel network at a level by weight of from about 0.05 wt % to about 14 wt %. For example, the fatty alcohol may be present in an amount ranging from about 1 wt % to about 10 wt %, and/or from about 6 wt % to about 8 wt %.

The fatty alcohols useful herein include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Non-limiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

Gel network preparation: A vessel is charged with water and the water is heated to about 74° C. Cetyl alcohol, stearyl alcohol, and SLES surfactant are added to the heated water. After incorporation, the resulting mixture is passed through a heat exchanger where the mixture is cooled to about 35° C. Upon cooling, the fatty alcohols and surfactant crystallized to form a crystalline gel network. Table 1 provides the components and their respective amounts for an example gel network composition.

TABLE 1
Gel network components

| Ingredient | Wt. % |
|---|---|
| Water | 78.27% |
| Cetyl Alcohol | 4.18% |
| Stearyl Alcohol | 7.52% |
| Sodium laureth-3 sulfate (28% Active) | 10.00% |
| 5-Chloro-2-methyl-4-isothiazolin-3-one, Kathon CG | 0.03% |

2. Emusifiers

A variety of anionic and nonionic emulsifiers can be used in the personal care composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

3. Chelating Agents

The personal care composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

Chelating agents can be incorporated in the compositions herein in amounts ranging from 0.001% to 10.0% by weight of the total composition, may be from 0.01% to 2.0%.

Nonlimiting chelating agent classes include carboxylic acids, aminocarboxylic acids, including aminocids, phosphoric acids, phosphonic acids, polyphosponic acids, polyethyleneimines, polyfunctionally-substituted aromatic, their derivatives and salts.

Nonlimiting chelating agents include the following materials and their salts. Ethylenediaminetetraacetic acid (EDTA), ethylenediaminetriacetic acid, ethylenediamine-N,N′-disuccinic acid (EDDS), ethylenediamine-N,N′-diglutaric acid (EDDG), salicylic acid, aspartic acid, glutamic acid, glycine, malonic acid, histidine, diethylenetriaminepentaacetate (DTPA), N-hydroxyethylethylenediaminetriacetate, nitrilotriacetate, ethylenediaminetetrapropionate, triethylenetetraaminehexaacetate, ethanoldiglycine, propylenediaminetetracetic acid (PDTA), methylglycinediacetic acid (MODA), diethylenetriaminepentaacetic acid, methylglycinediacetic acid (MGDA), N-acyl-N,N′,N′-ethylenediaminetriacetic acid, nitrilotriacetic acid, ethylenediaminediglutaric acid (EDGA), 2-hydroxypropylenediamine disuccinic acid (HPDS), glycinamide-N, N-disuccinic acid (GADS), 2-hydroxypropylenediamine-N—N′-disuccinic acid (HPDDS), N-2-hydroxyethyl-N,N-diacetic acid, glyceryliminodiacetic acid, iminodiacetic acid-N-2-hydroxypropyl sulfonic acid, aspartic acid N-carboxymethyl-N-2-hydroxypropyl-3-sulfonic acid, alanine-N,N′-diacetic acid, aspartic acid-N,N′-diacetic acid, aspartic acid N-monoacetic acid, iminodisuccinic acid, di amine-N,N′-dipoly acid, mono amide-N,N′-dipolyacid, diaminoalkyldi(sulfosuccinic acids) (DDS), ethylenediamine-N—N-bis (ortho-hydroxyphenyl acetic acid)), N,N′-bis (2-hydroxybenzyl)ethylenediamine-N, N-diacetic acid, ethylenediaminetetraproprionate, triethylenetetraaminehexacetate, diethylenetriaminepentaacetate, dipicolinic acid, ethylenedicysteic acid (EDC), ethylenediamine-N,N′-bis(2-hydroxyphenylacetic acid) (EDDHA), glutamic acid diacetic acid (GLDA), hexadentateaminocarboxylate (HBED), polyethyleneimine, 1-hydroxydiphosphonate, aminotri(methylenephosphonic acid) (ATMP), nitrilotrimethylenephosphonate (NTP), ethylenediaminetetramethylenephosphonate, diethylenetriaminepentamethylenephosphonate (DTPMP), ethane-1-hydroxydiphosphonate (HEDP), 2-phosphonobutane-1,2,4-tricarboxylic acid, polvphosphoric acid, sodium tripolyphosphate, tetrasodium diphosphate, hexametaphosphoric acid, sodium metaphosphate, phosphonic acid and derivatives, Aminoalkylen-poly(alkylenphosphonic acid), aminotri(1-ethylphosphonic acid), ethylenediaminetetra(1-ethylphosphonic acid), aminotri(1-propylphosphonic acid), aminotri(isopropylphosphonic acid), ethylenediaminetetra(methylenephosphonic acid) (EDTMP), 1,2-dihydroxy-3,5-disulfobenzene.

### Aqueous Carrier

The personal care compositions can be in the form of pourable liquids (under ambient conditions). Such compositions will therefore typically comprise a carrier, which is present at a level of from about 40% to about 85%, alternatively from about 45% to about 80%, alternatively from about 50% to about 75% by weight of the personal care composition. The carrier may comprise water, or a miscible mixture of water and organic solvent, and in one aspect may comprise water with minimal or no significant concentrations of organic solvent, except as otherwise incidentally incorporated into the composition as minor ingredients of other essential or optional components.

The carrier useful in the personal care compositions of the present invention may include water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

The azoxystrobin containing product may be a liquid, solid or powder or combinations thereof and can be dispensed from a container or can be a single use product. Non-limiting examples of single use products may include a discrete product that is in the form of a solid foam, capsule, pill, pod, sheet, film, tablet, compressed powder, encapsulated liquid, pouch or fibers. A powder may be dispensed from a container or delivered from an aerosol as a dry shampoo. The product may also be a liquid cleansing composition that is rinsed off including for cleansing skin or hair including shampoo, conditioners, body wash, or facial cleansing.

### pH

The personal care compositions mentioned above may also comprise one or more pH adjusting material. The compositions may have a pH in the range from about 2 to about 10, at 25° C. The rinse-off conditioner composition, and/or leave-on treatment may have a pH in the range of from about 2 to about 6, alternatively from about 3.5 to about 5, alternatively from about 5.25 to about 7.

The personal care compositions mentioned above may further comprise one or more pH buffering agent. Suitable buffering agents are well known in the art and include for example ammonia/ammonium acetate mixture and monoethanolamine (MEA). The rinse-off conditioner composition may comprise citric acid, wherein the citric acid acts as a buffer.

### Optional Ingredients

The personal care composition herein may optionally comprise one or more additional components known for use in personal care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt. % to about 10 wt. % by weight of the personal care compositions.

Non-limiting examples of additional components for use in the personal care compositions include conditioning agents, natural cationic deposition polymers, synthetic cationic deposition polymers, other anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, proteins, skin active agents, sunscreens, UV absorbers, and vitamins.

### 1. Conditioning Agent

The personal care compositions may comprise one or more conditioning agents. Conditioning agents include materials that are used to give a particular conditioning benefit to hair. The conditioning agents useful in the personal care compositions of the present invention typically comprise a water-insoluble, water-dispersible, non-volatile, liquid that forms emulsified, liquid particles. Suitable conditioning agents for use in the personal care composition are those conditioning agents characterized generally as silicones, organic conditioning oils or combinations thereof, or those conditioning agents which otherwise form liquid, dispersed particles in the aqueous surfactant matrix.

One or more conditioning agents are present from about 0.01 wt. % to about 10 wt. %, from about 0.1 wt. % to about 8 wt. %, and from about 0.2 wt. % to about 4 wt. %, by weight of the composition.

### Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers

### Organic Conditioning Materials

The conditioning agent of the compositions of the present invention may also comprise at least one organic conditioning material such as oil or wax, either alone or in combination with other conditioning agents, such as the silicones described above. The organic material can be nonpolymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-20 200, PEG-400, PEG-600, PEG-1000, PEG-2M, PEG-7M, PEG-14M, PEG-45M and mixtures thereof.

### Benefit Agents

The personal care composition may further comprise one or more additional benefit agents. The benefit agents comprise a material selected from the group consisting of anti-dandruff agents, anti-fungal agents, anti-itch agents, anti-bacterial agents, anti-microbial agents, moisturization agents, anti-oxidants, vitamins, lipid soluble vitamins, perfumes, brighteners, enzymes, sensates, attractants, dyes, pigments, bleaches, and mixtures thereof.

The personal care compositions of the present invention may be presented in typical personal care formulations. They may be in the form of solutions, dispersion, emulsions, powders, talcs, encapsulated, spheres, spongers, solid dosage forms, foams, and other delivery mechanisms. The compositions of the present invention may be hair tonics, leave-on hair products such as treatment, and styling products, rinse-off hair products such as hair conditioners, and treatment products; and any other form that may be applied to hair. The personal care composition may be a hair mask, cowash, hair wax, hair clay, hair food, hair milk, hair pudding and hair gels.

The personal care compositions may be provided in the form of a porous, dissolvable solid structure, such as those disclosed in U.S. Patent Application Publication Nos. 2009/0232873; and 2010/0179083, which are incorporated herein by reference in their entirety. Accordingly, the personal care compositions comprise a chelant, a buffer system comprising an organic acid, from about 23% to about 75% surfactant; from about 10% to about 50% water soluble polymer; and optionally, from about 1% to about 15% plasticizer; such that the personal care composition is in the form of a flexible porous dissolvable solid structure, wherein said structure has a Percent open cell content of from about 80% to about 100%.

The personal care compositions may be in the form of a porous dissolvable solid structure comprising a chelant; a buffer system comprising an organic acid from about 23% to about 75% surfactant; wherein said surfactant has an average ethoxylate/alkyl ratio of from about 0.001 to about 0.45; from about 10% to about 50% water soluble polymer; and from about 1% to about 15% plasticizer; and wherein said article has a density of from about 0.03 g/cm³ to about 0.20 g/cm³.

The personal care compositions may be in the form of a viscous liquid comprising a chelant; a buffer system comprising an organic acid from 5-20% surfactant and a polycarboxylate rheology modifier; wherein the polycarboxylate is specifically chosen to be effective at the high electrolyte levels resulting from the incorporation of the key buffer system and chelant used for this invention. Non-limiting examples include acrylates/C10-C30 alkyl acrylate crosspolymers such as Carbopol EDT2020, 1342,1382, etc. from Lubrizol. Rheology benefits of these actives may include stability, ease of dispensing, smoothness of spreading, etc.

The personal care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The personal care composition may be in a single phase or a single product, or the personal care composition may be in a separate phases or separate products. If two products are used, the products may be used together, at the same time or sequentially. Sequential use may occur in a short period of time, such as immediately after the use of one product, or it may occur over a period of hours or days.

Use of azoxystrobin in the personal care compositions of the present invention comprising one or more sulfate free surfactants may improve a dandruff condition. Use of azoxystrobin in the personal care composition of the present invention comprising one or more sulfate free surfactants may provide reduction of dandruff. Use of azoxystrobin in a personal care composition of the present invention comprising one of more sulfate free surfactants as claimed in present claim set may provide reduction of dandruff.

### Methods

In Vivo Fungal Efficacy Testing

Subjects from all test groups will have Baseline scalp swabs for measurement of scalp Malassezia. Subjects will take home a test product(s) and will be instructed on use test products throughout the week. The test concludes at week 1 or week 2 with panelists' scalps being swabbed and samples collected. Malassezia is quantified from scalp surface swabs via qPCR. The change in Malassezia amount across time will be reported as % fungal reduction from baseline at the 1- or 2-week time point.

### In Vivo Scalp Deposition Testing

The on-scalp deposition of the anti-dandruff active is measured by having the hair of individuals washed with a composition comprising an anti-dandruff active, for example a composition pursuant to the present invention. A trained cosmetician will dose the liquid shampoo control at 5 g on ½ of the panelist scalp and wash according to conventional washing protocol. Then 5 g of test shampoo is dosed to the other half of the panelist head and washed according to a conventional washing protocol. The hair is then parted on an area of the scalp to allow an openended glass cylinder to be held on the surface while an aliquot of an extraction solution is added and agitated prior to recovery and analytical determination of anti-dandruff active content by conventional methodology, such as HPLC.

### Measurement of Active Deposition

The concentration of the agent in the ethanol extraction solvent is measured by HPLC. Quantitation is made by reference to a standard curve. The concentration detected by HPLC is converted into an amount collected in grams by using the concentration multiplied by volume. The deposition efficiency can be calculated using the following equation. The area of the scalp extracted in each case is held constant:

Deposition efficiency=Mass agent deposited by example formula/Mass agent deposited by control formula

Sample calculation for deposition efficiency, where:
Mass of AZ deposited by example formula=1.0 ug
Mass of AZ deposited by control formula=0.5 ug

Deposition Efficiency=1.0/0.5

Deposition Efficiency=2X

### In-Vitro Fungal Inhibition Testing

The Zone of Inhibition (ZOI) methodology is chosen for this evaluation. In the ZOI method, Malassezia yeast organisms are seeded on a petri dish filled with growth medium. In this experiment, 15 μl of 1:100 diluted product is spotted onto the surface of culture plates, which are then incubated at 37° C. The applied product diffuses radially over time, with the anti-fungal potency indicated by the inhibition of fungal growth circularly from the center. The diameter of this circular inhibition is measured, the larger the circle, the more potent the anti-fungal activity of the product. Experiments use 5 replicates per leg and a t-test is performed at a significance level of 0.05.

### Minimum Inhibitory Concentration (MIC)

In Vitro Minimum Inhibitory Concentration (MIC) Testing

Malassezia furfur (CBS 7982) is maintained continuously as a culture at 31° C. in a 250-ml vent-capped polycarbonate Erlenmeyer flask by combining approximately 50 ml of mDixon growth medium and 2.5 ml of previously grown Malassezia culture. For each assay, Malassezia cells from 24-hour-old culture (approximately 7.5×10⁸ cells/ml) are diluted 500-fold into mDixon growth medium. Micropipettes are used to transfer 295 ul of diluted cells to each well of a Beckman 267007 polypropylene round-bottom deep-well plate.

Product forms are prepared for testing as concentrated stocks in water. Micropipettes are used to transfer 5 ul of appropriately diluted product form to the diluted Malassezia cells in the round-bottom deep-well plate. A semipermeable sealing film is applied to the plate which is then covered with water-soaked cotton batting. The deep-well plates are shaken at 31° C. on a Heidolph Titramax 1000 shaker at 1350 rpm for approximately 20 hours. The samples are mixed by micropipetting before transferring 200 ul of sample culture from each well to a Corning 3596 polystyrene plate. The plates are read immediately for absorbance at 600 nm using a Molecular Devices SpectraMax M5 plate reader. MIC values are presented as ppm of active.

### Stability

The stability of a composition is measured by placing samples of the composition at various temperatures for an extended period of time and then evaluating the sample for changes versus its target measure. Samples of the composition are placed at 5, 25, and 40 degrees Celsius for 3 months. The criteria for passing the viscosity stability evaluation is for the composition to retain its viscosity above 4000 centipoise. The criteria for passing the pH stability evaluation is for the composition to retain its pH within ±1 versus its target pH measure. The criteria for passing the appearance stability evaluation is for the composition to qualitatively retain the same appearance versus its target appearance measure.

### Preparation of the Control

Control compositions are prepared by creating a formulation with Azoxystrobin in sulfated surfactants. The formulation is adjusted to about pH 6. For example, the formulation shown as Example B is the control and fungal efficacy testing composition for the test composition A.

### Non-Limiting Examples

The shampoo compositions illustrated in the following examples are prepared by conventional formulation and mixing methods. All exemplified amounts are listed active wt. percent and exclude minor materials such as diluents, preservatives, color solutions, imagery ingredients, botanicals, and so forth, unless otherwise specified.

### Preparation of the Example Shampoo Compositions

The example cleaning compositions are prepared by combining the surfactant(s), polymers, the antidandruff active, preservatives, and the remainder of the water with ample agitation to ensure a homogenous mixture. The mixture can be heated to 65-75° C. to speed the solubilization of the surfactants, then cooled. Product pH is then adjusted as necessary to create thickening and resulting of approximately a pH of 5-7.

| Example Compositions | A | | B | |
|---|---|---|---|---|
| Sodium Laureth 3 Sulfate (SLE3S) ¹ | | | 8.00 | |
| Sodium Lauryl Sulfate ² | | | 7.00 | |
| Sodium Cocoyl Isethionate ³ | 6.00 | | | |
| Sodium Lauroyl Sarcosinate ⁴ | 4.00 | | | |
| Cocamidopropyl Betaine ⁵ | | | 2.00 | |
| Lauramidopropyl Betaine ⁶ | 9.75 | | | |
| Azoxystrobin ⁷ | 1.00 | | 1.00 | |
| Acrylate Copolymer ⁸ | 0.70 | | | |
| Guar Hydroxypropyltrimonium | | | 0.25 | |
| Chloride (HMW), 0.74 CD ⁹ | | | | |
| Polyquaternium-10~1.8Mil | 0.60 | | | |
| MW, 0.7 CD ¹⁰ | | | | |
| Fragrance | 0.85 | | 0.85 | |
| Dimethicone DC 1872 ¹¹ | 0.5 | | | |
| Dimethiconol ¹² | | | 0.80 | |
| Ethylene Glycol Distearate ¹³ | | | 1.50 | |
| Citric Acid ¹⁴ | Up to | | Up to | |
| | 2% | | 2% | |
| Methylchloroisothiazolinone/ | | | 5 | ppm |
| Methylisothiazolinone ¹⁵ | | | | |
| Sodium Salicylate ¹⁶ | 0.15 | | | |
| Sodium Chloride ¹⁷ | Up to | | Up to | |
| | 5% | | 5% | |
| Sodium Benzoate ¹⁸ | 0.25 | | 0.25 | |
| Tetrasodium EDTA ¹⁹ | | | 0.13 | |
| Water (q.s. to 100%) | q.s. | | q.s. | |
| pH | 5.7 | | 6.0 | |
| Deposition | 2.5 | ug/cm²= | 3.0 | ug/cm² |
| *Significantly up +, parity =, | | | Control | |
| Significantly down − | | | | |
| Deposition Efficiency | 0.83X | | Control | |
| In-Vitro Fungal Inhibition | 21 | mm= | 21 | mm |
| *Significantly up +, parity =, | | | Control | |
| Significantly down − | | | | |
| Minimum inhibitory Concentration | 0.123 | ppm= | 0.123 | ppm |

| | |
|---|---|
| *Significantly up +, parity =, | Control |
| Significantly down − | |

Key
¹ SLE3S, supplier: P&G Chemicals
² SLS 29% active, supplier: Stepan Company
³ Jordapon CI Prill at 84-89% active, supplier: BASF
⁴ Crodasinic LS-30NP at 30% active, supplier: Croda
⁵ Tego Betain L 7 OK at 30% active, supplier: Evonik
⁶ Mackam DAB ULS at 30% active, supplier: Solvay
⁷ Azoxystrobin, supplier: Nantong
⁸ Rheocare TTA at 30% active, supplier: BASF
⁹ N-Hance 3196, supplier: Ashland Specialty Ingredients
¹⁰ UCARE Polymer LR-30M, supplier: Dow Chemical
¹¹ Dimethicone DC 1872, supplier: Dow Chemical
¹² Belsil DM5500 at 42% active, supplier: Wacker
¹³ EGDS Purified, supplier: Evonik Goldschmidt Corporation
¹⁴ Citric Acid Anhydrous, supplier: Archer Daniels Midland
¹⁵ Kathon CG at 1.5% active, supplier: Rohm & Haas
¹⁶ Sodium Salicylate, supplier: JQC (Huayin) Pharmaceutical Co., Ltd.
¹⁷ Sodium Chloride, supplier: Morton
¹⁸ Sodium Benzoate Dense NF/FCC, supplier: Emerald Performance Materials
¹⁹ Dissolvine 220-S at 84% active, supplier: Akzo Nobel

### ZOI:

| Example Composition | ZOI (mm) |
|---|---|
| A | 21 |
| B | 21 |
| Commercial Anti-Dandruff Product with 1% | 13 |
| Potentiated ZPT | |

### Stability:

| Accelerated Stability | |
|---|---|
| Measure | |
| 3 Months (5 C., 25 C., 40 C.) | Composition A Result |
| Viscosity | PASS |
| pH | PASS |
| Appearance | PASS |

### Results

It has been surprisingly identified that composition A containing sulfate-free surfactants and 1% Azoxystrobin resulted in parity Azoxystrobin deposition vs. composition B control containing sulfated surfactants and 1% Azoxystrobin. In addition, it has been identified that Sulfate-free composition A has exhibited parity in-vitro Malassezia inhibition and minimum inhibitory concentration (MIC) vs. sulfated composition B control. When compared to a commercially available potentiated (i.e. with zinc carbonate) 1% ZPT (anti-dandruff composition), Sulfate-free composition A exhibited significantly higher Malassezia inhibition. Sulfate-free composition A with 1% Azoxystrobin has also exhibited 3 months of stability at temperatures ranging from 5 to 40 degrees Celsius.

### Examples and Compositions

The following examples illustrate non-limiting examples of the invention described herein. The exemplified shampoos, rinse-off conditioners, leave on treatments, personal care cleansing, single unit dose compositions can be prepared by conventional formulation and mixing techniques. It will be appreciated that other modifications of the oxidative dyeing compositions and rinse-off conditioner compositions within the skill of those in the formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

The following examples further describe and demonstrate non-limiting within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

| | Example, active wt % | | | |
|---|---|---|---|---|
| Ingredients | 1 | 2 | 3 | 4 |
| Sodium Lauroyl Sarcosinate ¹ | 5.0 | | 2.0 | 10.0 |
| Sodium Cocoyl Isethionate ² | 3.0 | | 6.0 | |
| Sodium Lauroyl Methyl Isethionate ³ | | 10.5 | | |
| Disodium Cocoamphodiacetate ⁴ | | 3.5 | | |
| Cocamidopropyl Betaine ⁵ | 4.0 | | 9.75 | 2.5 |
| Cocamide MEA ⁶ | | | | 1.0 |
| Azoxystrobin ⁷ | 0.1 | 0.25 | 0.5 | 1.0 |
| Guar Hydroxypropyltrimonium | 0.5 | 0.4 | | |
| Chloride ⁸ | | | | |
| Polyquaternium-10 ⁹ | | | 0.5 | 0.4 |
| Acrylates Copolymer ¹⁰ | 0.25 | 0.5 | 0.7 | 1.2 |
| Sodium Benzoate ¹¹ | 0.25 | 0.25 | 0.25 | 0.25 |
| Tetrasodium EDTA ¹² | 0.13 | 0.2 | 0.13 | 0.13 |
| Methylchloroisothiazolinone/ | 5 ppm | | | 5 ppm |
| Methylisothiazolinone ¹³ | | | | |
| Sodium Salicylate ¹⁴ | | 0.25 | 0.45 | |
| Citric Acid ¹⁵ | Up to | Up to | Up to | Up to |
| | 2% | 2% | 2% | 2% |
| Fragrance | 1.0 | 0.9 | 0.75 | 1.2 |
| Sodium Chloride ¹⁶ | Up to | Up to | Up to | Up to |
| | 3% | 3% | 3% | 3% |
| Water | q.s. | q.s. | q.s. | q.s. |
| PH | 6.0 | 5 | 5.5 | 6.5 |

¹ Crodasinic LS-30NP at 30% active, supplier: Croda
² Jordapon CI Prill at 84-89% active, supplier: BASF
³ Iselux at 80-85% active, supplier: Innospec
⁴ Miranol C2M Conc NP at 38.5%, supplier: Rhodia
⁵ Tego Betain L 7 OK at 30% active, supplier: Evonik
⁶ Ninol Comf at 85% active, supplier: Stepan
⁷ Azoxystrobin
⁸ N-Hance BF-17. supplier: Ashland Specialty Ingredients
⁹ UCARE Polymer KG-30M, supplier: Dow Chemical
¹⁰ Rheocare TTA at 30% active, supplier: BASF
¹¹ Sodium Benzoate Dense NF/FCC, supplier: Emerald Performance Materials
¹² Dissolvine 220-S at 84% active, supplier: Akzo Nobel
¹³ Kathon CG at 1.5% active, supplier: Rohm & Haas
¹⁴ Sodium Salicylate, supplier: JQC (Huayin) Pharmaceutical Co., Ltd.
¹⁵ Citric Acid Anhydrous, supplier: Archer Daniels Midland; level adjustable to achieve target pH
¹⁶ Sodium Chloride, supplier: Morton; level adjustable to achieve target viscosity

| | Example, active wt % | | | | | |
|---|---|---|---|---|---|---|
| Ingredients | 5 | 6 | 7 | 8 | 9 | 10 |
| Sodium Cocoyl Alaninate ¹ | 10.0 | | | | | |
| Sodium Cocoyl Isethionate ² | | 6.0 | | | | |
| Sodium Methyl Cocoyl Taurate ³ | | | 6.0 | 4.0 | | 10.0 |
| Sodium Caproyl Methyltaurate ⁴ | 2.0 | | 6.0 | | | |
| Lauramidopropyl Betaine ⁵ | | 9.75 | | 8.0 | | |
| Lauryl Hydroxysultaine ⁶ | | | | | | 1.0 |
| Disodium Cocoyl Glutamate ⁷ | | | | 4.0 | 16.0 | |
| Coco-glucoside ⁸ | | | | | | 1.0 |
| Decyl glucoside ⁹ | | | | | 4.0 | |
| Cocamide MEA ¹⁰ | 0.5 | | 1.0 | | | |
| Azoxystrobin ¹¹ | 0.75 | 0.5 | 1.2 | 1.0 | 0.25 | 0.5 |
| Guar Hydroxypropyltrimonium Chloride ¹² | 0.4 | | 0.5 | | 0.5 | 0.25 |
| Polyquaternium-10 ¹³ | | 0.55 | | 0.4 | | |
| Acrylates Copolymer ¹⁴ | 1.5 | | 2.5 | | 1.0 | |
| Ethylene Glycol Distearate ¹⁵ | 0.5 | | | 1.5 | 0.5 | 1.5 |
| Dimethiconol ¹⁶ | 0.5 | | 1.0 | | 0.8 | |
| Sodium Benzoate ¹⁷ | 0.1 | 0.25 | 0.25 | 0.1 | 0.45 | 0.25 |
| Tetrasodium EDTA ¹⁸ | 0.13 | 0.13 | 0.13 | 0.13 | 0.13 | 0.13 |
| Sodium Salicylate ¹⁹ | 0.25 | 0.45 | 0.25 | 0.25 | 0.25 | 0.25 |
| Citric Acid ²⁰ | Up to | Up to | Up to | Up to | Up to | Up to |
| | 2% | 2% | 2% | 2% | 2% | 2% |
| Fragrance | 1.2 | 0.9 | 0.75 | 1.0 | 0.5 | 0.8 |
| Sodium Chloride ²¹ | Up to | Up to | Up to | Up to | Up to | Up to |
| | 5% | 5% | 5% | 5% | 5% | 5% |
| Water | q.s. | q.s. | q.s. | q.s. | q.s. | q.s. |
| pH | 5.5 | 5.7 | 6.2 | 4.0 | 6.5 | 5.0 |

¹ Eversoft ACS-30S at 30% active, supplier: Sino Lion
² Jordapon CI Prill at 84-89% active, supplier: BASF
³ Pureact WS Cone at 30% active, supplier: Innospec
⁴ Diapon HF-SF at 27% active, supplier: NOF Corporation
⁵ Mackam DAB ULS at 30% active, supplier: Solvay
⁶ Mackam LHS at 41% active, supplier: Solvay
⁷ Eversoft UCS-50SG at 40% active, supplier: Sino Lion
⁸ Plantaren 818 UP at 50% active, supplier: BASF
⁹ Plantaren 2000 N UP at 50% active, supplier: BASF
¹⁰ Ninol Comf at 85% active, supplier: Stepan
¹¹ Azoxystrobin
¹² N-Hance 3196, supplier: Ashland Specialty Ingredients
¹³ UCARE Polymer LR-30M, supplier: Dow Chemical
¹⁴ Rheocare TTA at 30% active, supplier: BASF
¹⁵ EGDS Purified, supplier: Evonik Goldschmidt Corporation
¹⁶ Belsil DM5500 at 42% active, supplier: Wacker
¹⁷ Sodium Benzoate Dense NF/FCC, supplier: Emerald Performance Materials
¹⁸ Dissolvine 220-S at 84% active, supplier: Akzo Nobel
¹⁹ Sodium Salicylate, supplier: JQC (Huayin) Pharmaceutical Co., Ltd.
²⁰ Citric Acid Anhydrous, supplier: Archer Daniels Midland; level adjustable to achieve target pH
²¹ Sodium Chloride, supplier: Morton; level adjustable to achieve target viscosity

In the present invention, the personal care composition may comprising one or more sulfate free surfactants and 1% azoxystrobin results in parity azoxystrobin deposition when compared to a composition control comprising sulfated surfactants and 1% azoxystrobin. In the present invention the personal care composition may comprise one or more sulfate free surfactants and 1% azoxystrobin resulting in parity in-vitro Malassezia inhibition by minimum inhibitory concentration (MIC) when compared to a composition control comprising sulfated surfactants and 1% azoxystrobin. In the present invention, the personal care composition may comprise one or more sulfate free surfactants and 1% azoxystrobin resulting in parity in-vitro Malassezia inhibition by zone of inhibition (ZOI) when compared to a composition control comprising sulfated surfactants and 1% azoxystrobin. In the present invention, the personal care composition may comprise one or more sulfate free surfactants and 1% azoxystrobin results in a significantly higher Malassezia inhibition by zone of inhibition (ZOI) concentration when compared to a commercially marketed sulfated composition which is a potentiated composition comprising 1% zinc pyrithione.

### Methods of Making the Compositions

The formulations of the present invention may be present in typical personal care compositions. They may be in the form of solutions, dispersion, emulsions, powders, talcs, encapsulated, spheres, spongers, solid dosage forms, foams, and other delivery mechanisms. The composition of the present invention may be hair tonics, leave-on hair products such as conditioners, treatment, and styling products, and any other form that may be applied to the hair.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the personal care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular descriptions of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) from about 6% to about 50% of one or more sulfate free surfactants;
b) from about 0.02% to about 10% of azoxystrobin.

2. A personal care composition according to claim 1 wherein the one or more sulfate free surfactant are selected from the group consisting of sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

3. A personal care composition according to claim 1 comprising from about 0.05% to about 2% of azoxystrobin.

4. A personal care composition according to claim 1 wherein substantially free of a sulfate based surfactants is from about 0 wt % to about 3 wt %.

5. A personal care composition according to claim 1 wherein free of a sulfate based surfactant is 0 wt %.

6. A personal care composition according to claim 1 wherein the particle size of azoxystrobin is from about 0.5 microns to about 5 microns.

7. A personal care composition according to claim 4 wherein the particle size of azoxystrobin is from about 1 micron to about 3 microns.

8. A personal care composition according to claim 1 wherein the composition comprising one or more sulfate free surfactants and 1% azoxystrobin results in parity azoxystrobin deposition when compared to a composition control comprising sulfated surfactants and 1% azoxystrobin.

9. A personal care composition according to claim 1 wherein the composition comprising one or more sulfate free surfactants and 1% azoxystrobin results in parity in-vitro Malassezia inhibition by minimum inhibitory concentration (MIC) when compared to a composition control comprising sulfated surfactants and 1% azoxystrobin.

10. A personal care composition according to claim 1 wherein the composition comprising one or more sulfate free surfactants and 1% azoxystrobin results in parity in-vitro Malassezia inhibition by zone of inhibition (ZOI) when compared to a composition control comprising sulfated surfactants and 1% azoxystrobin.

11. A personal care composition according to claim 1 wherein the composition comprising one or more sulfate free surfactants and 1% azoxystrobin results in a significantly higher Malassezia inhibition by zone of inhibition (ZOI) concentration when compared to a commercially marketed sulfated composition which is a potentiated composition comprising 1% zinc pyrithione.

12. The personal care composition of claim 1, wherein the personal care composition further comprises one or more of a conditioning agent.

13. The personal care composition of claim 1, wherein said one or more conditioning agent is a silicone.

14. The personal care composition of claim 1, wherein the personal care composition further comprises a polymer.

15. The personal care composition of claim 14 wherein the polymer is a cationic polymer.

16. The personal care composition of claim 1, wherein said personal care composition further comprises one or more of a benefit agent.

17. The personal care composition according to claim 16 wherein the one or more benefit agent is selected from the group consisting of anti-dandruff agents, anti-fungal agents, anti-itch agents, anti-bacterial agents, anti-microbial agents, moisturization agents, anti-oxidants, vitamins, lipid soluble vitamins, perfumes, brighteners, enzymes, sensates, attractants, dyes, pigments, bleaches, and mixtures thereof.

18. A personal care composition according to claim 1 wherein the personal care composition is selected from the group consisting of a shampoo, rinse off conditioner, or a leave on treatment.

19. A personal care composition according to claim 1 further comprising from about 0.5% to about 7% of a perfume.

20. Use of azoxystrobin in a personal care composition comprising one or more sulfate free surfactants for improving a dandruff condition.

21. Use of azoxystrobin in a personal care composition comprising one or more sulfate free surfactants for reduction of dandruff.
